(19) United States
(12) Patent Application Publication
(10) Pub. No.: US 2018/0164327 A1
(43) Pub. Date: Jun. 14, 2018

(54) METHOD AND DEVICE FOR TESTING RENAL FUNCTION USING URINARY VEGF-A165b AS INDICATOR, AND PROGRAM AND RECORDING MEDIUM FOR CAUSING TO FUNCTION AS DEVICE FOR TESTING RENAL FUNCTION

(71) Applicant: National University Corporation Nagoya University, Aichi (JP)

(72) Inventors: Ryosuke KIKUCHI, Aichi (JP); Tadashi MATSUSHITA, Aichi (JP); Toyoaki MUROHARA, Aichi (JP)

(21) Appl. No.: 15/577,702

(22) PCT Filed: May 24, 2016
(86) PCT No.: PCT/JP2016/065325

§ 371 Date: Nov. 28, 2017

(30) Foreign Application Priority Data
May 29, 2015 (JP) 2015-110113

(51) Int. Cl. G01N 33/68

(52) U.S. Cl. CPC G01N 33/6893; G01N 2800/52; G01N 2800/56; G01N 2800/347

(57) ABSTRACT

An object of the present embodiment is to provide a testing method by which it is possible to test for reduced renal function even in an early stage. The problem can be solved by a method for testing renal function, wherein the VEGF-A₁₆₅b content of urine is measured and the measured content is used as an indicator.

### BACKGROUND OF THE INVENTION

### 1. Field of the Invention

The present disclosure relates to a method and device for testing renal function, and a program and recording medium for causing to function as a device for testing renal function, and more particularly relates to a method and device that make it possible to perform early testing of reduced renal function by measuring the urinary content of VEGF-A₁₆₅b (vascular endothelial growth factor-A₁₆₅b) and using the measured content as an indicator, and to a program and recording medium for causing a computer to function as a device for testing renal function.

### 2. Description of the Related Art

Chronic kidney disease (CKD), which is now a national disease, progresses to end stage renal failure and must be treated by dialysis requiring high medical-care costs. In Japan, it is estimated that about 1 in 8 adults suffer from CKD, and patients required dialysis is said to be about 1 in 440.

Also, peripheral artery disease (PAD) is observed in 25.3% of dialysis patients according to the Dialysis Outcomes and Practice Patterns Study (DOPPS), which is an international forward-looking observational study of about 30,000 cases of hemodialysis patients. In recent years, it has become apparent that renal disorder is an important risk factor in arteriosclerotic disease, but renal disorder is also an important risk factor in PAD in which arteriosclerosis is the pathogenesis, and PAD is known to be a complicating factor in the high rate of renal disorder.

The seriousness (progression) of CKD is evaluated in stages related to cause, renal function, and urinary protein, and an appropriate treatment that corresponds to the stage is required. Renal function is categorized into six groups, namely, G1, G2, G3a, G3b, G4, and G5, depending on the estimated glomerular filtration rate (eGFR), and the seriousness is higher as the eGFR decreases. There are almost no subjective symptoms in the G1 and G2 periods, which are initial categories.

On the other hand, subjective symptoms begin to appear in G3a and thereafter, and specialized treatment becomes required in G3a and thereafter. In the G3a and G3b periods, recovery can be expected with suitable exercise, no smoking, reduced alcohol consumption, dietetic treatment with reduced sodium, and other daily life improvements, or with reduced blood pressure by prescription of an angiotensin receptor blocker (ARB), calcium channel blocker (CCB), or other hypotensive agent. However, progression to G4 results in a condition prior to introduction of dialysis, and progression to G5 requires dialysis. Ultimately, renal transplant or the like is required, and in the current state, it is difficult to recover renal function to a normal condition. Also, life prognosis after introduction of dialysis is very poor in CKD. Consequently, early discovery is very important for treatment of CKD.

Known methods for testing for CKD test for biomarkers, i.e., β2 microglobulin in urine, which is a biological sample obtained by a noninvasive collection method, and small amounts of serum albumin (hereinafter referred to as “microalbumin”) discharged in urine without being reabsorbed at sites referred to as renal glomeruli (see non-patent documents 1 to 3).

### PRIOR ART DOCUMENTS

### Non-Patent Documents

[Non-patent document 1] Richard J. Glassock., “Is the Presence of Microalbuminuria a Relevant Marker of Kidney Disease?”, Curr Hypertens Rep. 2010, 12(5):364-368
[Non-patent document 2] Yue et al., “Urinary biomarkers to detect acute kidney injury in the pediatric emergency center.”, Pediatr Nephrol (2011)26:267-274
[Non-patent document 3] Jill et al., “Urinary and serum biomarkers for the diagnosis of acute kidney injury: an in-depth review of the literature.”, Nephrol Dial Transplant (2012)0:1-20

### SUMMARY OF THE INVENTION

As described above, early discovery is important for CKD. However, β2 microglobulin is an acute marker for renal tubular disorders, but is particularly unstable in acidic urine. Consequently, it is necessary to adopt measures such as evaluating voluntary urine a plurality of times in clinical settings and using the highest values, and there is a problem in that testing methods are laborious. Furthermore, there is a problem in that concentration of β2 microglobulin in urine increases due also to β2 microglobulin production caused by a malignant tumor or the like. On the other hand, with microalbumin in urine, when the function of the filtration membrane in the kidney is degraded, proteins that are no longer filtered out then leak out in small amounts. Consequently, measuring the ratio of albumin in urinary protein in very small amounts makes it possible to evaluate abnormal renal function, but there is a problem with sensitivity. As described above, when β2 microglobulin and microalbumin are used as biomarkers, there is a problem in that evaluation is difficult until renal function has reached a certain state of degradation. Accordingly, there is a need for a detection method capable of detection for early CKD with good sensitivity using urine, which is a noninvasive biological sample, but currently, there are no known biomarkers capable of detection with good sensitivity.

The present disclosure was devised in order to solve the problems with the prior art as described above, and after thoroughgoing studies, the present disclosure was perfected after it was newly found that (1) there is a correlation between the VEGF-A₁₆₅b in urine and eGFR, (2) since there is a correlation between VEGF-A₁₆₅b in urine and eGFR, these can become biomarkers which can be used for testing for reduction of renal function in an early period, (3) renal function is reduced in commensurate fashion to reduced VEGF-A₁₆₅b content in urine, (4) comparing the VEGF-A₁₆₅b content and a reference value allows the stage of renal function to be determined, and (5) the progress of CKD can be tested in an early period because deterioration in renal function can be tested in an early stage.

In other words, an object of the present disclosure is to provide a method and device for testing renal function using VEGF-A₁₆₅b in urine as an indicator, and a program and recording medium for causing a computer to function as a device for testing renal function.

The present disclosure, as shown below, relates to a method and device for testing renal function using VEGF-A₁₆₅b in urine as an indicator, and a program and recording medium for causing a computer to function as a device for testing renal function.

(1) A method for testing renal function, wherein

a VEGF-A₁₆₅b content in urine is measured and the measured content is used as an indicator.

(2) The method for testing of (1) above, wherein

a renal function is determined to deteriorate commensurately with respect to a decrease in the VEGF-A₁₆₅b content.

(3) The method for testing of (1) or (2) above, wherein

the measured content is compared with a reference value, and a stage of renal function is determined.

(4) The method for testing of any of Claims (1) to (3) above, wherein the measured content is a value corrected using results of measuring creatinine components in urine.

(5) A device for testing renal function, comprising at least:

a storage device for saving a VEGF-A₁₆₅b content in urine measured in advance and a reference value set on the basis of the stage of renal function;

an input device for inputting a measured VEGF-A₁₆₅b content in urine of a test subject; and

a computation device for comparing the measured content inputted using the input device and the reference value stored in the storage device, and thereby determining the stage of renal function.

(6) The device for testing renal function of (5) above, wherein at least information related to the VEGF-A₁₆₅b content in urine measured in advance and to the stage of renal function are stored in the storage device, and the reference value can be set and/or modified on the basis of the stored information.

(7) A program for causing a computer to function as the device for testing renal function of (5) or (6) above.

(8) A computer-readable recording medium in which the program of (7) above is recorded.

### Effects of the Invention

In accordance with the present disclosure, using VEGF-A₁₆₅b contained in urine, which is a noninvasive biological sample, as a biomarker makes it possible to carry out testing for reduced renal function earlier than with conventional biomarkers. Therefore, testing of renal function is simple, early stage CKD patients can be discovered, and therefore an appropriate treatment method can be selected.

Also, discovering early stage CKD patients makes it possible to reduce the number of patients to be started on dialysis, which requires high treatment costs, and to reduce medical-care costs.

Furthermore, it is possible to automate the testing of renal function in which progress is in an early period by providing a testing device comprising storage device for saving reference values set on the basis of the VEGF-A₁₆₅b content in urine and computation device for comparing the VEGF-A₁₆₅b content in urine of a test subject with a reference value stored in the storage device and thereby determining the stage of renal function, and providing a program and recording medium for causing a computer to function as a device for testing renal function.

### BRIEF DESCRIPTION OF THE DRAWINGS

FIG. 1 schematically shows a test device;

FIG. 2 shows the steps for determining stages of renal function using the test device 1 of the present embodiment;

FIG. 3 is a chart showing the relationship between the eGFR values and VEGF-A₁₆₅b values obtained in example 1, and a table representing the number of specimens categorized into stages, and the average value, standard deviation, and standard error of the VEGF-A₁₆₅b of the specimens in each stage;

FIG. 4 represents ROC curves created on the basis of the results calculated in example 1;

FIG. 5 is a chart and table showing the relationship between β2 microglobulin values and eGFR values obtained in comparative example 1;

FIG. 6 represents ROC curves created on the basis of the results calculated in comparative example 1;

FIG. 7 is a chart and table showing the relationship between microalbumin values and eGFR values obtained in comparative example 2; and

FIG. 8 represents ROC curves created on the basis of the results calculated in comparative example 2.

### DESCRIPTION OF THE PREFERRED EMBODIMENTS

Described in detail below are the method and device for testing renal function using VEGF-A₁₆₅b in urine as an indicator, and the program and recording medium for causing a computer to function as a device for testing renal function of the present embodiments.

The method for testing renal function of the present embodiment uses the VEGF-A₁₆₅b content in urine as an indicator. VEGF-A (vascular endothelial growth factor) is known as a group of glycoproteins involved in angiogenesis and neovascularization. VEGF-A binds as a ligand to vascular endothelial growth factor receptors (VEGFR), which are mainly located on the surface of vascular endothelial cells, and work to stimulate cell division, migration, and differentiation, or to accelerate vascular permeability of microvasculature. VEGF-A is known to additionally be involved in activation of monocytes and macrophages, and is also known to be associated with neovascularization of a normal body, as well as to be involved in processes of malignant alteration such as tumor angiogenesis and metastasis.

On the other hand, VEGF-A₁₆₅b, which is in the VEGF-A family, was identified in 2002 as an isoform in which a portion of the sequences generated by specific splicing at the terminus of the 8th exon, which is a constituent element of the VEGF-A gene, are different (David O. B. et. al., “VEGF165b, an Inhibitory Splice Variant of Vascular Endothelial Growth Factor, Is Down-Regulated in Renal Cell Carcinoma.”, Cancer Res. 2002 Jul. 15; 62(14):4123-31). VEGF-A₁₆₅b is known to have the effect of inhibiting VEGF-A-induced vascular endothelial proliferation, epithelial migration, vasodilation, in vivo angiogenesis, and tumor growth.

Regarding the problem in which ischaemia of lower limb tissues in not improved in spite of the fact that in-blood concentration of VEGF-A, which causes new blood vessels to be created in PAD patients, has increased more predominately than with a normal subject, the present inventors found that, by distinguishing and measuring neovascularization-promoting VEGF-A₁₆₅a and neovascularization-inhibiting VEGF-A₁₆₅b, VEGF-A₁₆₅b increases in PAD patients and this is a factor in the cause of anangioplasia (R. Kikuchi et. Al., “An antiangiogenic isoform of VEGF-A contributes to impaired vascularization in peripheral artery disease”, Nature Medicine 20, P.1464-1471, (2014)). However, the use of VEGF-A₁₆₅b in urine as a biomarker for testing renal function is not known.

The VEGF-A₁₆₅b content in urine can be quantified by a known method for quantifying protein. For example, quantification is possible by ELISA (enzyme-linked immunosorbent assay), Western blot (WB), immunoprecipitation/Western blot protocol (IP-WB), or other technique using an anti-VEGF-A₁₆₅b antibody. Quantification is also possible by LC-MS or the like.

It is possible to calculate eGFR using the formula

eGFR (mL/minute/1.73 m²)=194×Cr^{-1.094}×age^{-0.287}(×0.739 for females),

wherein Cr represents serum creatinine, which can be measured using a commercially available kit or the like.

The sensitivity and specificity of the measured results can be confirmed using a receiver operating characteristic curve (ROC curve). An ROC curve is used when evaluating precision in screening tests and the like and when comparing conventional testing and novel testing, and shows the range in which a cutoff point should be selected. Selecting a cutoff point makes it possible to visually show the ability of a test to distinguish a person having a condition (disease) from one who does not. An ROC curve plots the true positive rate (specificity) on the vertical axis and the false positive rate (1-specificity) on the horizontal axis. The cutoff point to be used are determined from the resulting curve considering the seriousness of the disease, the positioning of testing, and various other conditions. However, when the cutoff point is a point with a low false positive rate, the number of normal subjects determined to be positive is reduced, but many subjects having a disease would be excluded. When sensitivity is conversely increased, the false positive rate is increased. When the superiority or inferiority of different tests is to be determined, the curve is capable of determining superiority the closer a result is positioned to the upper left. For example, when the curve of a new testing technique is further to the upper left than the ROC curve of a conventional testing technique, the new testing technique can be determined to have greater precision and to be superior. An ROC curve can be determined by analyzing measurement results using known software.

In the present embodiment, comparing the measured VEGF-A₁₆₅b content in urine with a reference value makes it possible to determine reduced (stage) renal function. CKD is defined in the “Clinical Practice Guidebook for Diagnosis and Treatment of Chronic Kidney Disease 2012” to be G3a or a later stage among G1 to G5, which is one reference for evaluating renal function. In other words, CKD can be determined when G3a or a later stage has been determined as a result of testing renal function using the method and device for testing renal function of the present embodiment. Consequently, the method and device for testing renal function of the present embodiment can also be used as a method and device for testing for CKD without special modification. Therefore, in the present embodiment, “the method and device for testing renal function” also means “method and device for testing for CKD.” In the “Clinical Practice Guidebook for Diagnosis and Treatment of Chronic Kidney Disease 2012,” G1 to G5 and CKD are categorized into the following six stages using the eGFR value calculated using the formula shown above.

G1(90≤eGFR) 1.

Renal function is estimated to be normal or at a high value, and is not CKD.

G2(60≤eGFR<90) 2.

Renal function is estimated to be normal or mildly reduced, and is not CKD.

G3a(45≤eGFR<60) 3.

Renal function is estimated to be mildly or intermediately reduced, and CKD is suspected.

G3b(30≤eGFR<45) 4.

Renal function is estimated to be intermediately or greatly reduced, and CKD is strongly suspected.

G4(15≤eGFR<30) 5.

Renal function is estimated to be greatly reduced; this stage is CKD, and there is a high possibility of complications with various abnormalities (anemia, mineral abnormalities, bone abnormalities, and the like) caused by reduced renal function.

G5(eGFR<15) 6.

Terminal renal failure is estimated. This indicates a state directly prior to the need for dialysis or other treatment.

The measured value of the VEGF-A₁₆₅b content can be used without modification, or may also be a value corrected using the result of measuring creatinine components in urine. The concentration of components in urine is generally affected by diet, water intake, sweating, and the like, and considerably fluctuates depending on the amount of urine at the time. In other words, component concentrations may possibly differ depending on the concentration of urine. On the other hand, creatinine in urine is thought to be substantially constant regardless of disease in a single body because the production of creatinine depends on the amount of muscle. Consequently, in a test of the secreted substance in urine, a technique is generally used in which the amount of target secreted substance in the urine is corrected by the amount of creatinine per gram in order to avoid error, and it is thereby possible to compare the secreted substance in urine per unit gram of creatinine.

In the present embodiment, it was newly found that there is a correlation between VEGF-A₁₆₅b in urine and eGFR, and therefore, renal function (CKD) can be tested by using the VEGF-A₁₆₅b content in urine as an indicator. Consequently, in the case of the same test subject, it is possible to periodically measure the VEGF-A₁₆₅b content in urine, and determine whether reduction of renal function is progressing based on whether the measured content is decreasing.

Also, (1) it is possible to provide a device for testing renal function (CKD) that determines the stage of renal function (CKD) by collecting the urine of many CKD patients and healthy subjects in advance to measure the VEGF-A₁₆₅b content in the urine, saving in the storage device the measured content and a reference value set on the basis of the renal function stage, and comparing the VEGF-A₁₆₅b content in the urine of a test subject with the reference value (hereinafter merely referred to as “testing device”).

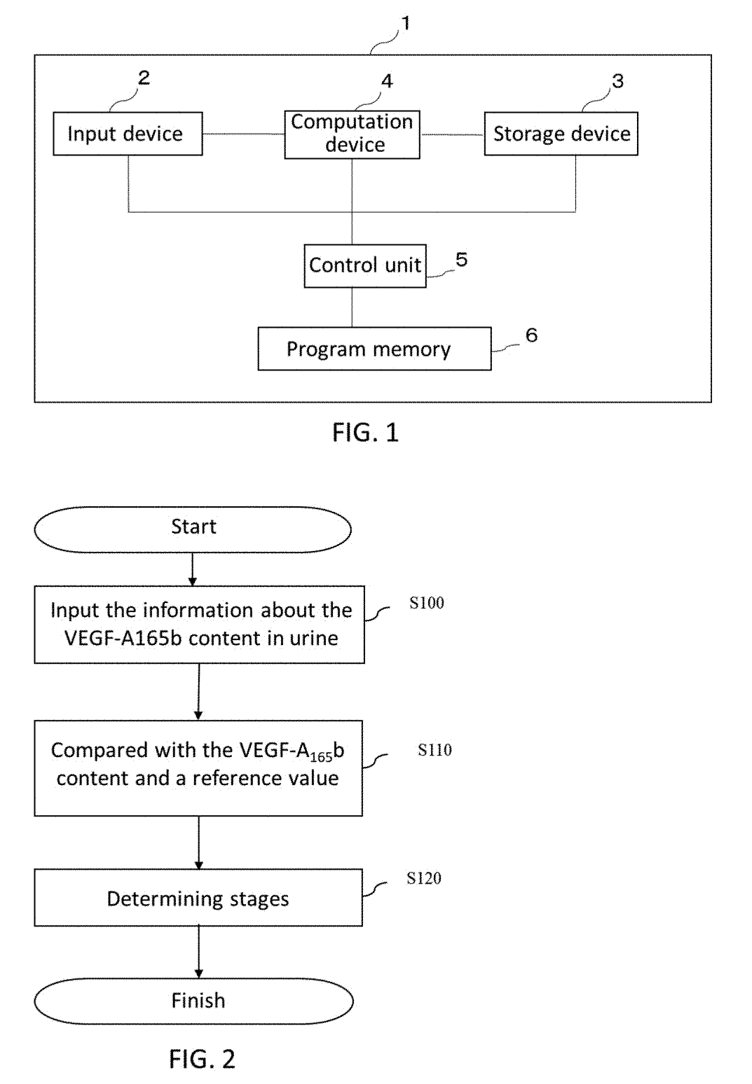

FIG. 1 is a diagram schematically showing the test device. The test device 1 includes at least input device 2, storage device 3 in which a reference value is saved, computation device 4 for determining the stage of renal function by comparing the VEGF-A₁₆₅b content in the urine of a test subject inputted using the input device 2 and the reference value stored in the storage device 3, a control unit 5, and a program memory 6. Display device, a printer (not shown), and/or other device for outputting measurement results may also be included.

The input device 2 is not particularly limited as long as information about the VEGF-A₁₆₅b content in the urine of a test subject can be inputted to the test device 1; examples including a keyboard, USB, or the like. An Internet connection may also be used as the input device 2. For example, it is possible to transmit/input information about the measurement results acquired in a remote hospital using an Internet connection to the test device 1, send the measurement results via the Internet connection, and thereby appropriately determine the stage of renal function (CKD) for a patient in the remote hospital. It is also possible to connect the test device 1 and an automated analysis device capable of analyzing components in a urine sample, and automatically input the analysis results produced by the automated analysis device to the test device 1 to determine the stage and thereby automate urine sample analysis and determination.

The storage device 3 is not particularly limited as long as a reference value can be stored as described above. The reference value can be modified by positioning testing, seriousness of disease and various other conditions. Therefore, VEGF-A₁₆₅b and eFGR obtained from the urine of a large number of CKD patients and healthy subjects, and the serum creatinine content, stage of renal function, and other information are also stored in the storage device 3, and the reference value can be set and/or modified, as appropriate, on the basis of the information stored in the storage device 3. The reference value is not particularly limited as long as renal function (CKD) can be determined. For example, it is possible set a single reference value, and determine whether the stage of a test subject is G3a or higher, G4 or higher, or another stage. It is also possible to provide a plurality of reference values, and determine which stage such as G3a or G5 to which a test subject corresponds.

The computation device 4 compares the information about the content inputted using the input device 2 and the reference value stored in the storage device 3, and is thereby able to determine the stage of renal function (CKD). Saved in program memory 6 is, e.g., a program for causing a computer to function as the test device 1 shown in FIG. 1. The program is read out and executed by the control unit 5, whereby operational control of the input device 2, storage device 3, and computation device 4 is carried out. The program may be stored in a computer in advance, and may be stored in a recording medium together with the reference value and/or the VEGF-A₁₆₅b obtained from the urine of a large number of patients and healthy subjects, the eFGR, the serum creatinine content, the stage of renal function, and other information; and saved in program memory 6 using installing means.

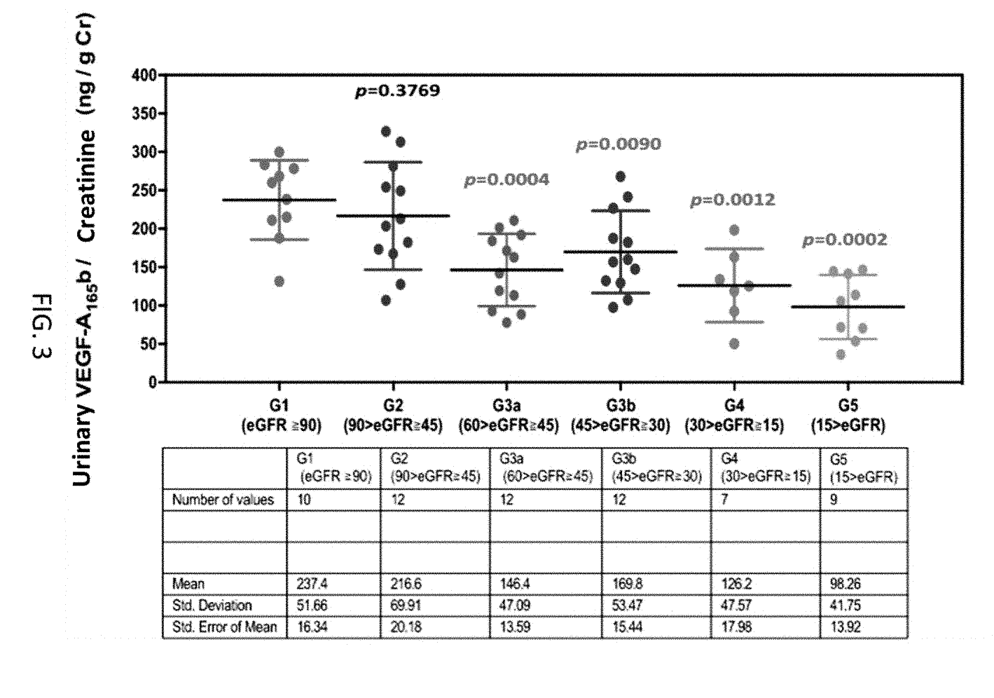

FIG. 2 is a diagram showing the steps for determining stages of renal function (CKD) using the test device 1 of the present embodiment. The program saved in the program memory 6 is read out and executed by the control unit 5, and first information about the VEGF-A₁₆₅b content in the urine of a test subject is inputted using the input device 2 (S100). As described above, the VEGF-A₁₆₅b content in the urine may be a value corrected using urinary creatinine as required. Next, information about the VEGF-A₁₆₅b content inputted using the input device 2 is compared with the reference value stored in the storage device 3 (S110). The determined stage of renal function is then displayed (S120). Determination may be made as a stage of renal function, or determination may be made as a stage of CKD (determined not to be CKD in the case of G1 or G2). The display method may be by display on computer display means or on paper or another printout.

The present embodiment is described in detail below using examples, which are provided for reference to a detailed mode in order to describe the present embodiment in a simple manner. These examples are for describing a specific detailed mode of the present embodiment, and do not limit or represent a limitation of the scope of the invention disclosed in the present application.

### EXAMPLES

Specimens, measurement of components in urine, and the method for analyzing measurement results which are used in examples and comparative examples are described below.

<Clinical Specimens>

Urine specimens stored in the Nagoya University Hospital, Department of Clinical Laboratory were used with the approval of Nagoya University Hospital Bioethics Committee (Approval No.: 1038). In the present example, 62 urine specimens were used.

<Measurement of the VEGF-A₁₆₅b Content in Urine>

The VEGF-A₁₆₅b content in urine was measured using Human Vascular Endothelial Growth Factor-A₁₆₅b ELISA Kit (MyBioSource Inc.: MBS720132)

<Measurement of Serum Creatinine, Urinary Creatinine, Microalbumin in Urine, and β2 Microglobulin in Urine>

Serum creatinine and urinary creatinine were measured using a creatinine kit for blood and urine testing (Cygnus Auto CRE: Shino-Test Corp.), microalbumin in urine was measured using “serotec” TIA-ALBG for measuring albumin in urine (serotec Co., Ltd.), β2 microglobulin in urine was measured using Beta 2-microglobulin kit BMG-Latex X1 “Seiken” (DENKA SEIKEN Co., Ltd.), and content was measured using Hitachi Automatic Analyzer LABOSPECT0008 (Hitachi High-Tech Fielding Corporation).

<Urinary Creatinine Correction of Measurement Results>

Correction of the measured components in urine was carried out using the measurement results of urinary creatinine in the same urine.

VEGF-A₁₆₅b Value

The content (ng) of VEGF-A₁₆₅b per gram of urinary creatinine

Microalbumin Value

The content (mg) of microalbumin per gram of urinary creatinine

β2 Microglobulin Value

The β2 microglobulin content (μg) per gram of urinary creatinine

<Sensitivity and Specificity Test by ROC Curve>

An ROC curve was created using GraphPad Prism 6 software for statistical analysis. The Mann-Whitney test was used as the statistical technique for testing the difference between two independent groups. The result was determined to be statistically significant at p<0.05.

<Calculation of eGFR>

The eGFR was calculated using the measured value for serum creatinine and formula below.

eGFR (mL/minute/1.73 m²)=194×Cr^{-1.094}×age^{-0.287}(×0.739 for females).

<Evaluation of Renal Function>

Renal function was categorized into the six stages of G1 (90≤eGFR), G2 (60≤eGFR<90), G3a (45≤eGFR<60), G3b (30≤eGFR<45), G4 (15≤eGFR<30), and G5 (eGFR<15) on the basis of the calculated eGFR.

### Example 1

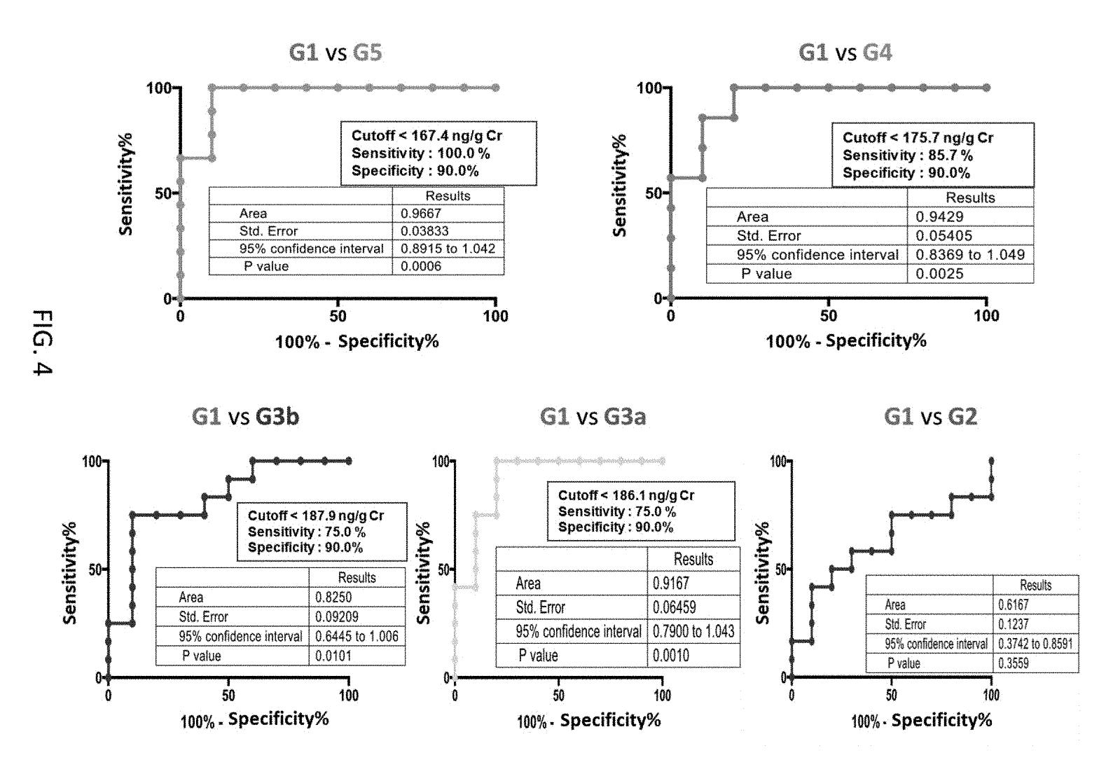

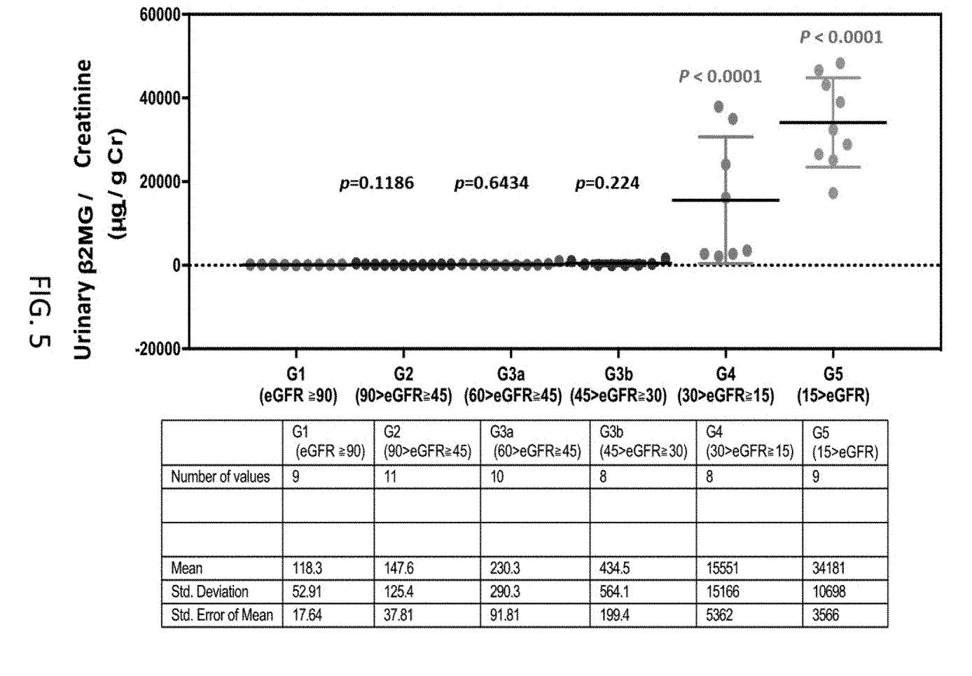

The eGFR value and the VEGF-A₁₆₅b value in 62 urine specimens were calculated using the above-described method. Next, the 62 specimens were categorized into G1, G2, G3a, G3b, G4, and G5 on the basis of the eGFR values, and the VEGF-A₁₆₅b values were plotted. FIG. 3 is a chart showing the relationship between the VEGF-A₁₆₅b values and eGFR values obtained in example 1. The horizontal bar of each stage represents, in sequence from the top, the upper quartile point, median value, and lower quartile point. The p values noted in G2 to G5 are values obtained by comparison with G1. The table below the plot of FIG. 3 shows the number of specimens categorized into the stages G1 to G5, and the average value, standard deviation, and standard error of the VEGF-A₁₆₅b of the specimens in each stage. FIG. 4 represents ROC curves created on the basis of the results calculated in example 1.

### Comparative Example 1

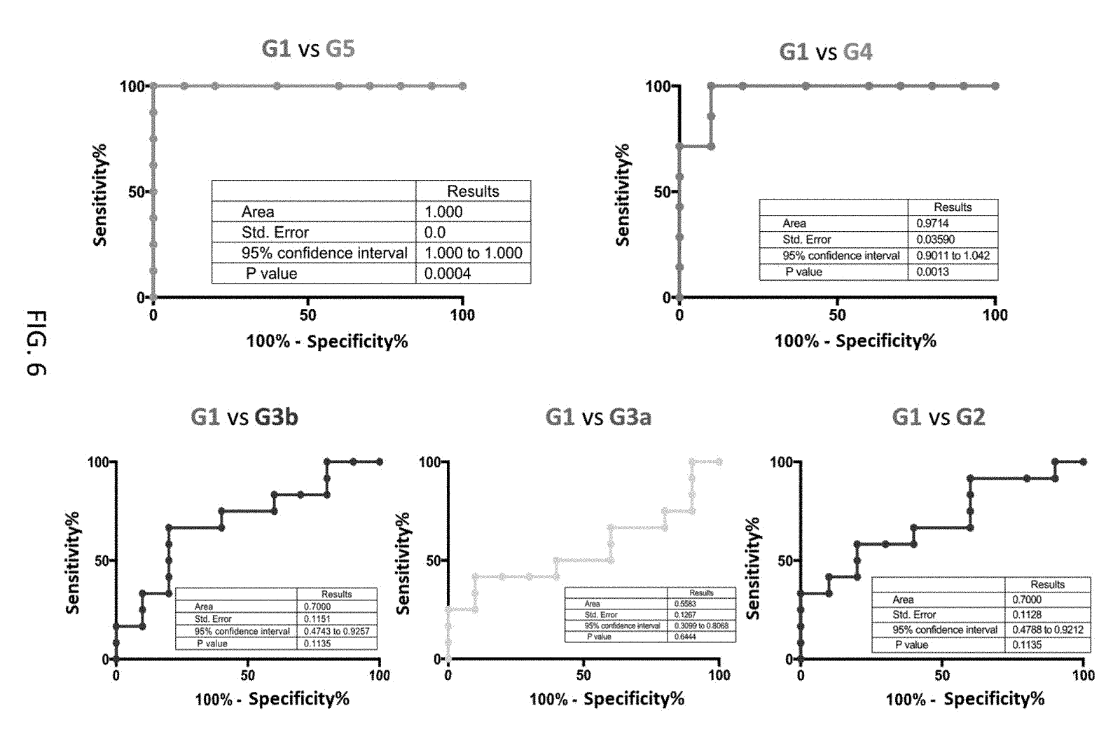

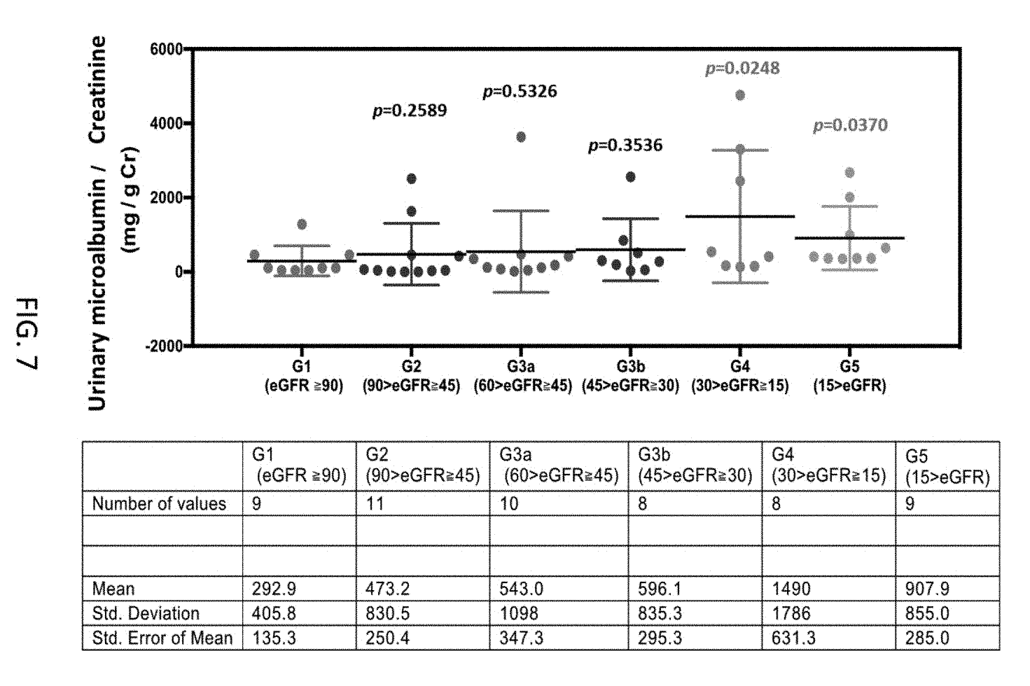

Plotting was carried out using the same procedure as in example 1, except that β2 microglobulin values were used in place of the values of VEGF-A₁₆₅b in urine. FIG. 5 shows a chart and table created to show the relationship between β2 microglobulin values and eGFR values. FIG. 6 represents ROC curves created on the basis of the results calculated in comparative example 1.

### Comparative Example 2

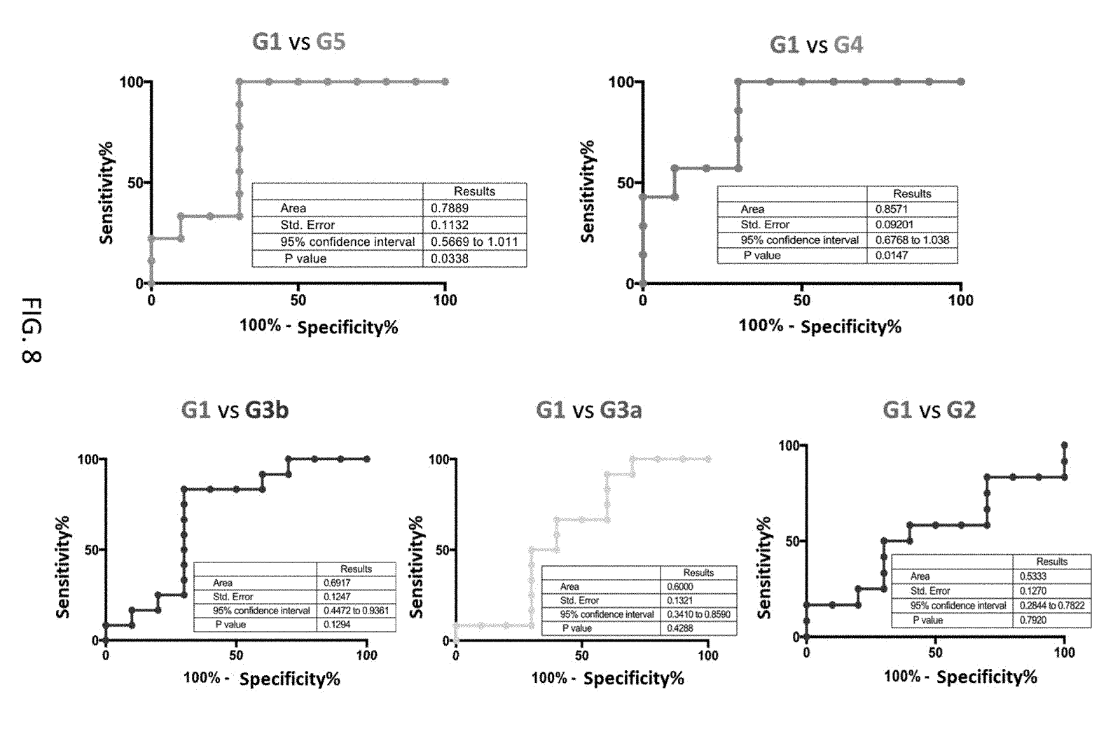

Plotting was carried out using the same procedure as in example 1, except that microalbumin values were used in place of the values of VEGF-A₁₆₅b in urine. FIG. 7 shows a chart and table created to show the relationship between microalbumin values and eGFR values. FIG. 8 represents ROC curves created on the basis of the results calculated in comparative example 2.

As shown in FIG. 3, it was found that eGFR, which is an indicator for when the stages of renal function (CKD) are categorized, and the VEGF-A₁₆₅b content in urine in Example 1 are correlated. It was also found that the VEGF-A₁₆₅b content in urine is reduced as renal function degrades.

The p value of G1 vs G2 was 0.3559 and a significant difference was not observed when VEGF-A₁₆₅b was used as an indicator as shown by the ROC curve of FIG. 4. On the other hand, for G1 vs G3a, the p value was 0.001 and statistically significant when the cutoff value of the VEGF-A₁₆₅b content was set to <186.1 ng/gCr. For G1 vs G3b, the p value was 0.101 and statistically significant when the cutoff value of the VEGF-A₁₆₅b content was set to <187.9 ng/gCr. Furthermore, the likelihood ratio (sensitivity/(1-specificity)) was 7.5 for both G1 vs G3a and G1 vs G3b, the likelihood ratio being an indicator that represents the plausibility of a result when a test is positive. In the field of clinical testing, this value is a numerical value which can be used to determine that usefulness is high. From the above-described results, it was found that using the VEGF-A₁₆₅b content in urine as an indicator makes it possible to test a patient having renal function categorized as G3a or higher (patients having early CKD) with high sensitivity and specificity.

On the other hand, in comparative example 1 in which the β2 microglobulin value was used as an indicator, and as shown in FIGS. 5 and 6, sensitivity and specificity were higher than VEGF-A₁₆₅b when renal function that had progressed to CKD was stage G4 or G5, but detection was not possible in stages G3a and G3b. In comparative example 2 in which the microalbumin value was used as an indicator, sensitivity and specificity were low in the G4 and G5 periods, and detection was not possible in stages G3a and G3b, as shown in FIGS. 7 and 8. Furthermore, it is apparent from a comparison of the ROC curves in FIGS. 4, 6, and 8 that the ROC curves of G3a and G3b, in which VEGF-A₁₆₅b was used as an indicator shown in FIG. 4, were positioned further to the upper left than the ROC curves of G3a and G3b in which β2 microglobulin and microalbumin were used as indicators.

It was found from the results described above that VEGF-A₁₆₅b in urine provides higher and superior precision as a biomarker for detecting the G3a stage of renal function, i.e., the progress of CKD at an early stage, in comparison with β2 microglobulin and microalbumin, which are biomarkers of CKD that have conventionally been used.

### INDUSTRIAL APPLICABILITY

Measuring VEGF-A₁₆₅b content in urine allows renal function to be tested with good sensitivity and specificity at stage G3a, i.e., the progress of CKD at an early stage. Therefore, the present embodiment is useful for the medical care industry in that CKD patients can be provided appropriate treatment.

1-7. (canceled)

8. A method for testing renal function, wherein
a VEGF-A₁₆₅b content in urine is measured and the measured content is used as an indicator.

9. The method for testing of claim 8, wherein
a renal function is determined to deteriorate commensurately with respect to a decrease in the VEGF-A₁₆₅b content.

10. The method for testing of claim 8, wherein
the measured content is compared with a reference value, and a stage of renal function is determined.

11. The method for testing of claim 9, wherein
the measured content is compared with a reference value, and a stage of renal function is determined.

12. The method for testing of claim 8, wherein the measured content is a value corrected using results of measuring creatinine components in urine.

13. The method for testing of claim 9, wherein the measured content is a value corrected using results of measuring creatinine components in urine.

14. The method for testing of claim 10, wherein the measured content is a value corrected using results of measuring creatinine components in urine.

15. A device for testing renal function, comprising at least:
a storage device for saving a VEGF-A₁₆₅b content in urine measured in advance and a reference value set on the basis of the stage of renal function;
an input device for inputting a measured VEGF-A₁₆₅b content in urine of a test subject; and
a computation device for comparing the measured content inputted using the input device and the reference value stored in the storage device, and thereby determining the stage of renal function.

16. The device for testing renal function of claim 15, wherein at least information related to the VEGF-A₁₆₅b content in urine measured in advance and to the stage of renal function are stored in the storage device, and the reference value can be set and/or modified on the basis of the stored information.

17. A program for causing a computer to function as the device for testing renal function of claim 15.

18. A program for causing a computer to function as the device for testing renal function of claim 16.

19. A computer-readable recording medium in which the program of claim 17 is recorded.

20. A computer-readable recording medium in which the program of claim 18 is recorded.
